(19) United States
(12) Patent Application Publication
(10) Pub. No.: US 2022/0287257 A1
(43) Pub. Date: Sep. 15, 2022

(54) HIGH PROTEIN PEA

(71) Applicant: EQUI-nom Ltd., Givat Brenner (IL)

(72) Inventors: Gil SHALEV, Ramot Meir (IL); Noa PALEVSKY, Rehovot (IL); Menachem SKLARZ, Beer Sheva (IL); Avichai AMRAD, Kfar Bilu (IL); Sigal MEIROVITCH, Tel-Aviv (IL)

(73) Assignee: EQUI-nom Ltd., Givat Brenner (IL)

(21) Appl. No.: 17/630,126

(22) PCT Filed: Aug. 19, 2021
(86) PCT No.: PCT/IL2021/051020

§ 371 Date: Jan. 25, 2022

(30) Foreign Application Priority Data
Aug. 21, 2020 (CA) 3090792

(51) Int. Cl. A01H 1/00; A01H 5/10; A01H 6/54; A23J 3/22; A23J 3/14

(52) U.S. Cl. CPC A01H 1/108; A01H 5/10; A01H 6/546; A23J 3/227; A23J 3/14

(57) ABSTRACT

Uses and pea plant cells of pea plants and parts thereof, which contain higher protein than current varieties, are provided. Phenotypic and genotypic analysis of many pea varieties was performed to derive markers for high protein and other phenotypic traits, and a breeding simulation was used to identify the most common and most stable markers. Following verification of trait stability over several generations, markers and marker cassettes were defined as being uniquely present in the developed pea lines. The resulting high protein pea lines can be used to enhance the nutritional values of pea in its various uses. Uses include processing the seeds to yield any of pea protein isolate, pea concentrate, a texturized product, a meat analog and/or commodity whole or split grains.

### 1. TECHNICAL FIELD

The present invention relates to the field of pea genetics and breeding, and more particularly, to quantitative trait loci (QTLs) associated with seed protein content in pea.

### 2. DISCUSSION OF RELATED ART

Pea (Pisum sativum) is a cool season legume grown worldwide as a source of protein both for human food and animal feed. Economically, legumes represent the second most important family of crop plants, and dry pea currently ranks second only to common bean as the most widely grown grain legume in the world. Its primary production is in temperate regions. In 2018, its global production was 34.7 M tons.

Although pea is considered to be one of the world's oldest domesticated crops, classical breeding methodology attempts done in order to increase protein level encountered some obstacles due to inferior agronomical traits such as low yield potential. Commercial dry pea varieties, which currently grown in France and Canada, have an average protein content of about 22%. The plant protein market is constantly challenged by the growing worldwide demand for non-GMO plant-based protein. An increase in protein level represents a significant financial gain to protein processors and food companies, therefore there is a great need for improved pea lines and breeding methods for high seed protein level.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the plurality of QTLs and corresponding markers comprise at least three QTLs and corresponding markers, the QTL and marker associated with the semi-leafless trait comprise QTL 7 with corresponding markers set forth in Seq. IDs 13 or 14, and the pea plant or part thereof is homozygous with respect to Seq. ID 13 or heterozygous at QTL 7.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant, a part thereof or pea seeds having high protein content and obtained from said pea plant cell, wherein: the phenotypic traits comprise a high protein content of the seeds of at least 25% and semi-leafless and powdery mildew resistance, the plurality of QTLs and corresponding markers comprise at least three QTLs and corresponding markers, the QTL and marker associated with the semi-leafless trait comprise QTL 7 with corresponding markers set forth in Seq. IDs 13 or 14, and the pea plant cell is homozygous with respect to Seq. ID 13 or heterozygous at QTL 7.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the QTL and marker associated with the high protein trait comprise QTL 1 with corresponding marker set forth in Seq. IDs 1 or 2, the pea plant or part thereof comprise QTL 2 with corresponding marker set forth in Seq. IDs 3 or 4, the QTL and marker associated with the semi-leafless trait comprise QTL 7 with corresponding markers set forth in Seq. IDs 13 or 14, the QTL and marker associated with the powdery mildew resistance trait comprise QTL 9 with corresponding markers set forth in Seq. IDs 17 or 18, the pea plant or part thereof is homozygous with respect to Seq. ID 2 or heterozygous at QTL 1, the pea plant or part thereof is homozygous with respect to Seq. ID 3 or heterozygous at QTL 2, the pea plant or part thereof is homozygous with respect to Seq. ID 13 or heterozygous at QTL 7, and the pea plant or part thereof is homozygous with respect to Seq. ID 18 or heterozygous at QTL 9.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the QTL and marker associated with the high protein trait comprise QTL 1 with corresponding marker set forth in Seq. IDs 1 or 2, the pea plant or part thereof comprise QTL 2 with corresponding marker set forth in Seq. IDs 3 or 4, the QTL and marker associated with the semi-leafless trait comprise QTL 7 with corresponding markers set forth in Seq. IDs 13 or 14, the QTL and marker associated with the powdery mildew resistance trait comprise QTL 8 with corresponding markers set forth in Seq. IDs 15 or 16, the pea plant or part thereof is homozygous with respect to Seq. ID 1 or heterozygous at QTL 1, the pea plant or part thereof is homozygous with respect to Seq. ID 4 or heterozygous at QTL 2, the pea plant or part thereof is homozygous with respect to Seq. ID 13 or heterozygous at QTL 7, and the pea plant or part thereof is homozygous with respect to Seq. ID 15 or heterozygous at QTL 8.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the QTL and marker associated with the high protein trait comprise QTL 3 with corresponding marker set forth in Seq. IDs 5 or 6; and QTL 4 with corresponding marker set forth in Seq. IDs 7 or 8, the QTL and marker associated with the semi-leafless trait comprise QTL 7 with corresponding markers set forth in Seq. IDs 13 or 14, the QTL and marker associated with the powdery mildew resistance trait comprise QTL 8 with corresponding markers set forth in Seq. IDs 15 or 16, the pea plant or part thereof is homozygous with respect to Seq. ID 5 or heterozygous at QTL 3, the pea plant or part thereof is homozygous with respect to Seq. ID 7 or heterozygous at QTL 4, the pea plant or part thereof is homozygous with respect to Seq. ID 13 or heterozygous at QTL 7, and the pea plant or part thereof is homozygous with respect to Seq. ID 15 or heterozygous at QTL 8.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the QTL and marker associated with the high protein trait comprise QTL 5 with corresponding marker set forth in Seq. IDs 9 or 10, the pea plant or part thereof comprise QTL 6 with corresponding marker set forth in Seq. IDs 11 or 12, the QTL and marker associated with the semi-leafless trait comprise QTL 7 with corresponding markers set forth in Seq. IDs 13 or 14, the pea plant or part thereof is homozygous with respect to Seq. ID 9 or heterozygous at QTL 5, the pea plant or part thereof is homozygous with respect to Seq. ID 11 or heterozygous at QTL 6, and the pea plant or part thereof is homozygous with respect to Seq. ID 13 or heterozygous at QTL 7.

Some aspects of the present invention relate to uses of the pea plant or a part thereof, e.g., as textured vegetable products (TVPs), e.g., meat replacements, possibly having above 60% protein, above 65% protein or above 70% protein.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant obtained from said pea plant cell, wherein: the phenotypic traits comprise a high protein content of the seeds of at least 25% and semi-leafless and powdery mildew resistance, the QTL and marker associated with the high protein trait comprise QTL 1 with corresponding marker set forth in Seq. IDs 1 or 2, the pea plant cell comprises QTL 2 with corresponding marker set forth in Seq. IDs 3 or 4, the QTL and marker associated with the semi-leafless trait comprise QTL 7 with corresponding markers set forth in Seq. IDs 13 or 14, the QTL and marker associated with the powdery mildew resistance trait comprise QTL 9 with corresponding markers set forth in Seq. IDs 17 or 18, the pea plant cell is homozygous with respect to Seq. ID 2 or heterozygous at QTL 1, the pea plant cell is homozygous with respect to Seq. ID 3 or heterozygous at QTL 2, the pea plant cell is homozygous with respect to Seq. ID 13 or heterozygous at QTL 7, and the pea plant cell is homozygous with respect to Seq. ID 18 or heterozygous at QTL 9.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant, a part thereof or pea seeds having high protein content and obtained from said pea plant cell, wherein: the phenotypic traits comprise a high protein content of the seeds of at least 25% and semi-leafless and powdery mildew resistance, the QTL and marker associated with the high protein trait comprise QTL 1 with corresponding marker set forth in Seq. IDs 1 or 2, the pea plant cell comprises QTL 2 with corresponding marker set forth in Seq. IDs 3 or 4, the QTL and marker associated with the semi-leafless trait comprise QTL 7 with corresponding markers set forth in Seq. IDs 13 or 14, the QTL and marker associated with the powdery mildew resistance trait comprise QTL 8 with corresponding markers set forth in Seq. IDs 15 or 16, the pea plant cell is homozygous with respect to Seq. ID 1 or heterozygous at QTL 1, the pea plant cell is homozygous with respect to Seq. ID 4 or heterozygous at QTL 2, the pea plant cell is homozygous with respect to Seq. ID 13 or heterozygous at QTL 7, and the pea plant cell is homozygous with respect to Seq. ID 15 or heterozygous at QTL 8.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant, a part thereof or pea seeds having high protein content and obtained from said pea plant cell, wherein: the phenotypic traits comprise a high protein content of the seeds of at least 25% and semi-leafless and powdery mildew resistance, the QTL and marker associated with the high protein trait comprise QTL 3 with corresponding marker set forth in Seq. IDs 5 or 6; and QTL 4 with corresponding marker set forth in Seq. IDs 7 or 8, the QTL and marker associated with the semi-leafless trait comprise QTL 7 with corresponding markers set forth in Seq. IDs 13 or 14, the QTL and marker associated with the powdery mildew resistance trait comprise QTL 8 with corresponding markers set forth in Seq. IDs 15 or 16, the pea plant cell is homozygous with respect to Seq. ID 5 or heterozygous at QTL 3, the pea plant cell is homozygous with respect to Seq. ID 7 or heterozygous at QTL 4, the pea plant cell is homozygous with respect to Seq. ID 13 or heterozygous at QTL 7, and the pea plant cell is homozygous with respect to Seq. ID 15 or heterozygous at QTL 8.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant, a part thereof or pea seeds having high protein content and obtained from said pea plant cell, wherein: the phenotypic traits comprise a high protein content of the seeds of at least 25% and semi-leafless and powdery mildew resistance, the QTL and marker associated with the high protein trait comprise QTL 5 with corresponding marker set forth in Seq. IDs 9 or 10, the pea plant cell comprises QTL 6 with corresponding marker set forth in Seq. IDs 11 or 12, the QTL and marker associated with the semi-leafless trait comprise QTL 7 with corresponding markers set forth in Seq. IDs 13 or 14, the pea plant cell is homozygous with respect to Seq. ID 9 or heterozygous at QTL 5, the pea plant cell is homozygous with respect to Seq. ID 11 or heterozygous at QTL 6, and the pea plant cell is homozygous with respect to Seq. ID 13 or heterozygous at QTL 7.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIG. 1 is a high-level schematic illustration of pea chromosomes with indications of the markers' loci, according to some embodiments of the invention.

FIGS. 2A-2C present experimental results indicating the higher protein content and varying protein composition traits in pea varieties with the disclosed marker cassettes, according to some embodiments of the invention.

FIG. 3 is a high-level schematic illustration of the breeding method, according to some embodiments of the invention.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Uses and pea plant cells of pea plants and parts thereof, which contain higher protein than current varieties, are provided. Phenotypic and genotypic analysis of many pea varieties was performed to derive markers for high protein and other phenotypic traits, and a breeding simulation was used to identify the most common and most stable markers. Following verification of trait stability over several generations, markers and marker cassettes were defined as being uniquely present in the developed pea lines. The resulting high protein pea lines can be used to enhance the nutritional values of pea in its various uses. Uses include processing the seeds to yield any of pea protein isolate, pea concentrate, a texturized product, a meat analog or meat replacement and/or commodity whole or split grains. Certain embodiments comprise use of pea plants, parts thereof and/or pea seeds, which may be processed, as animal feed.

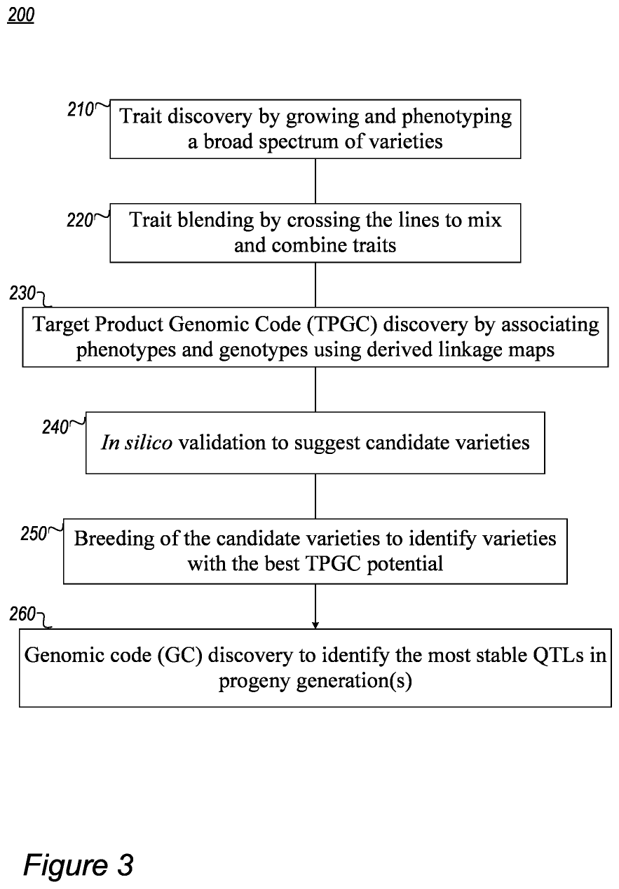

Various embodiments comprise pea cells and uses of pea plants or part(s) thereof that have high protein content and comprise a plurality of loci associated with a corresponding plurality quantitative trait loci (QTLs) having a corresponding plurality of nucleic acid genetic markers that are associated with a plurality of phenotypic traits of the pea plant. The phenotypic traits comprise a high protein content of the seeds of at least 25% and semi-leafless and powdery mildew resistance traits, and the plurality of QTLs and corresponding markers comprise at least two QTLs and corresponding markers—with details provided in Table 1 below. The methods used to develop and select the varieties are disclosed with respect to FIG. 3 below.

Various uses include processing the seeds to yield pea protein isolate and/or pea concentrate which provide the pea protein at different levels of concentration and with different amounts of additional compounds. The seeds may be processed into texturized products which may have mechanical properties in addition to their nutritional properties, e.g., texturized products may make a food product more firm or more crispy. The seeds may be processed into meat analogs to provide nutritional properties, chemical characteristics and similar look and feel (e.g., texture, flavor, appearance) as various types of meat. The seeds may be processed into commodity whole or split grains, possibly by drying or otherwise modifying the seeds.

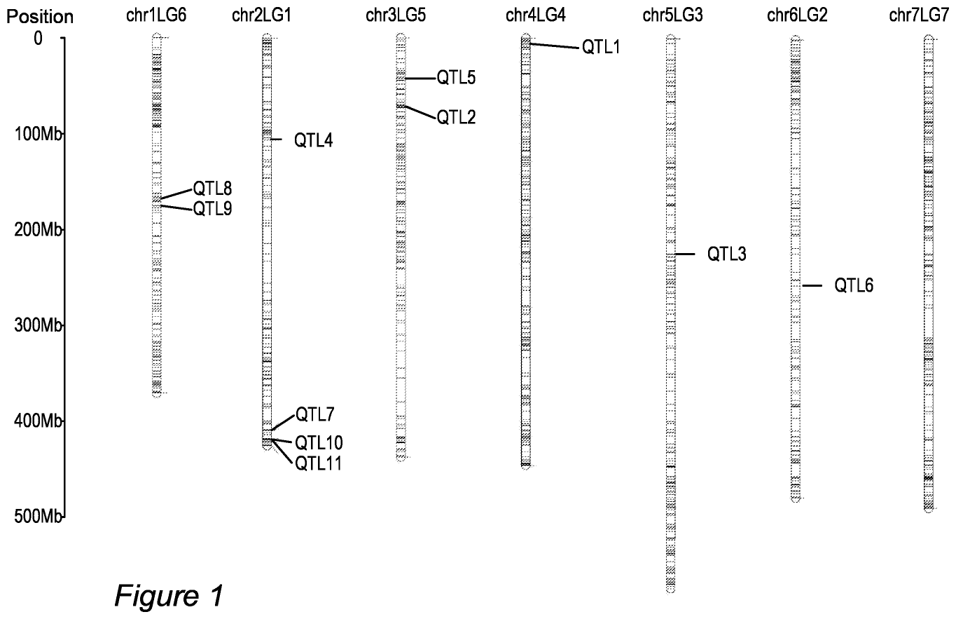

FIG. 1 is a high-level schematic illustration of pea chromosomes (and linkage groups—LGs) with indications of the markers' loci (QTL number), according to some embodiments of the invention. FIG. 1 illustrates schematically the seven pea chromosomes with their banding patterns, and the marker locations indicated along them.

Table 1 provides the derived genetic markers, QTLs, corresponding traits and resulting marker cassettes, according to some embodiments of the invention.

Table 2 provides protein content and composition data for plant varieties with the marker cassettes, according to some embodiments of the invention—compared to control varieties.

TABLE 1
Genetic markers, QTLs, corresponding traits and marker cassettes
with corresponding protein content and composition data.

| | Seq | | | | | |
|---|---|---|---|---|---|---|
| QTL | ID | Marker¹ | Chr/LG | Position | QTL P-value | Trait |
| 1 | 1, 2 | 038887_23884_703 | chr4LG4 | 6445332 | 0.033 | Protein content |
| 2 | 3, 4 | 017135_10651_5316 | chr3LG5 | 71669603 | 0.034 | Protein component |
| 3 | 5, 6 | 050373_32960_3169 | chr5LG3 | 225883023 | 0.018 | Protein content |
| 4 | 7, 8 | 029308_17474_1688 | chr2LG1 | 106604303 | 0.001 | Protein content |
| 5 | 9, 10 | 044073_28004_2765 | chr3LG5 | 44420741 | 0.001 | Protein content |
| 6 | 11, 12 | 07_32684348 | chr6LG2 | 259389351 | 0.013 | Protein component |
| 7 | 13, 14 | 42662_26712_871 | chr2LG1 | 410200645 | Qualitative | Semi-Leafless |
| 8 | 15, 16 | 044504_28363_461 | chr1LG6 | 167946502 | markers | Powdery mildew |
| 9 | 17, 18 | ER1 | chr1LG6 | 175515672 | | resistance |
| 10 | 19, 20 | 044835_28587_1878 | chr2LG1 | 419557580 | | Yellow |
| 11 | 21, 22 | 044855_28602_1561 | chr2LG1 | 419560368 | | cotyledon² |

| | | |
|---|---|---|
| | | Cassette (with |
| Seq | Allele | respective QTLs)³ |

| QTL | ID³ | 1 | 2 | 1 | 2 | 3 | 4 |
|---|---|---|---|---|---|---|---|
| 1 | 1, 2 | T | G | GG/GT | TT/TG | | |
| 2 | 3, 4 | A | G | AA/AG | GG/GA | | |
| 3 | 5, 6 | A | G | | | AA/AG | |
| 4 | 7, 8 | T | G | | | TT/TG | |
| 5 | 9, 10 | A | C | | | | AA/AC |
| 6 | 11, 12 | A | T | | | | AA/AT |
| 7 | 13, 14 | T | C | TT/TC | TT/TC | TT/TC | TT/TC |
| 8 | 15, 16 | T | C | | TT/TC | TT/TC | |
| 9 | 17, 18 | C | G | GG/GC | | | |
| 10² | 19, 20 | T | G | TT/TG | TT/TG | TT/TG | TT/TG |
| 11 ² | 21, 22 | A | G | AA/AG | AA/AG | AA/AG | AA/AG |

QTLs

| Cassette | Obligatory | Optional |
|---|---|---|
| 1 | 1, 2, 7, 9 | 10, 11 |
| 2 | 1, 2, 7, 8 | 10, 11 |
| 3 | 3, 4, 7, 8 | 10, 11 |
| 4 | 5, 6, 7 | 10, 11 |

¹The markers are as provided by Tayeh et al. 2015, except of 07_32684348 derived from independent RNA sequence analysis, and except of ER1 provided by Humphry et al. 2011.
²QTLs 10 and 11 for the yellow cotyledon trait are optional.
² QTLs10 and 11 are optional for the respective cassettes.
³For each QTL, the two Seq IDs represent the sequences for each allele; the zygosity (homozygous or heterozygous combination) is provided by the respective cassette entries.

TABLE 2
Protein content and composition data for plant varieties with the marker cassettes.

| | | | | | | Protein composition | | |
|---|---|---|---|---|---|---|---|---|
| | | | | | | (average, relative)¹ | | |
| | Protein content (%) | | | Dunnett's test 0.05 | | | | P-value |
| Plants type (n) | Average | Max | Min | Abs(Dif)-LSD | p-Value | Vicilin 6 | Vicilin 5 | (ANOVA) |
| Cassette 1 (5) | 26.52 | 27.18 | 25.75 | 2.442 | <.0001* | 1.7198 | | 0.0021 |
| Cassette 2 (12) | 26.88 | 28.59 | 25.84 | 2.134 | <.0001* | | 1.10018 | 0.025 |
| Cassette 3 (5) | 26.76 | 27.44 | 26.37 | 1.898 | <.0001* | | | |
| Cassette 4 (2) | 26.4 | 26.81 | 25.98 | 1.381 | 0.0002* | | | |
| Control (5) | 23.41 | 24.56 | 22.43 | −1.22 | 1 | 1.19003 | 1.65058 | |

¹Protein composition includes Vicilin traits 6 and 5 relating to QTL2, for cassettes 1 and 2, respectively. Further studies are underway.

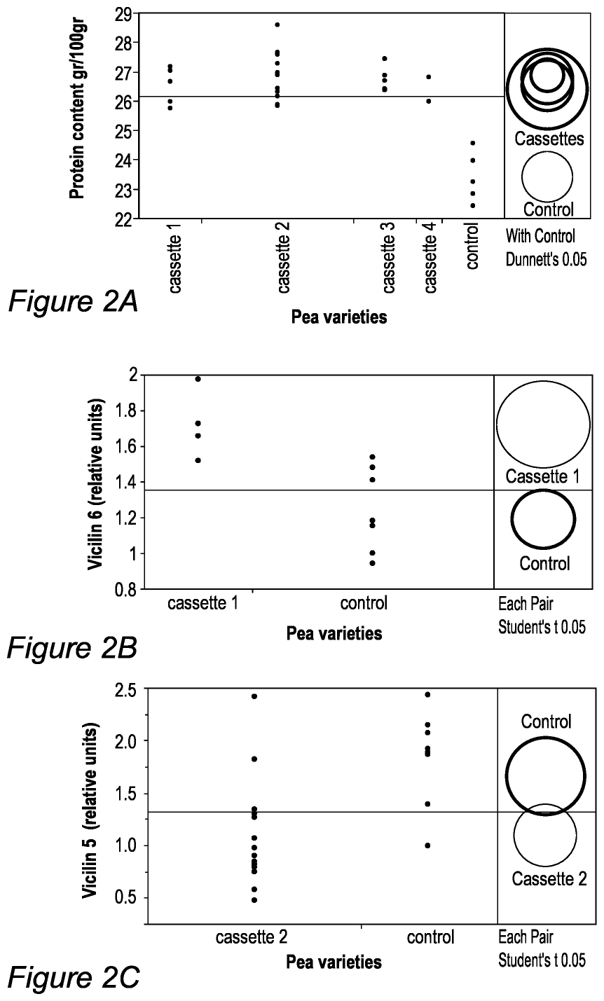

FIGS. 2A-2C present experimental results indicating the higher protein content and varying protein composition traits in pea varieties with the disclosed marker cassettes, according to some embodiments of the invention.

As indicated in Table 2 and illustrated in FIG. 2A, all four cassettes yield average protein content of at least 25% which is significantly distinct from prior art varieties that have lower protein content. FIG. 2A illustrates this significant difference graphically with respect to the multiple lines that were examined for each cassette (denoted by “n” in Table 2).

FIGS. 2B and 2C illustrate schematically the significant differences in protein composition of cassette 1 and cassette 2 varieties, with the former having significantly higher vicilin 6 content than the prior art varieties, and the latter having significantly lower vicilin 5 content than the prior art varieties. It is noted that Vicilin is one of the two major groups of storage proteins (Globulins) present in pea, together with Legumin (and Convicilin) it accounts for about 65-70% of the total protein content in the seed. Vicilin subunits are trimers, that form 6 subunits of 12-50 kDa. Vicilin 5 and 6 have the lowest molecular weight out of the subunits— 18 and 16 kDa respectively. All vicilin subunits are characterized by relatively high glycosylation, which contributes to their polarity and thus high water solubility and functionality. Accordingly, the inventors note that differences in protein composition may be related to the nutritional value and the processability of the resulting pea crop.

Disclosed QTLs comprise one or more of QTLs 1 to 11 with corresponding pairs of Seq IDs 1-22 that specify the alleles (with respective different SNP—Single Nucleotide Polymorphism—bases) of the respective markers that are linked to QTLs 1-11. It is noted that any of QTLs may be homozygous—having two identical alleles of the same Seq ID; or any of QTLs may be heterozygous having two different alleles with different Seq ID of each pair—as listed in Table 1 and below.

QTL 1, as used herein, refers to a polymorphic genetic locus linked to genetic marker 038887_23884_703 in pea linkage group 4 (LG4) on chromosome 4. The two alleles of marker 038887_23884_703 at QTL 1 have the SNP bases “T” or “G”, respectively, at position 6445332 of LG4, as set forth, respectively, in the nucleic acid sequences of Seq IDs 1 and 2. In cassette 1, QTL 1 may be homozygous for allele 2 (Seq ID 2) or be heterozygous (Seq IDs 1 and 2); while in cassette 2, QTL 1 may be homozygous for allele 1 (Seq ID 1) or be heterozygous (Seq IDs 1 and 2).

(SNP base bold):
Seq ID No. 1
CTTTCTTCTGTATTTCCTTCTTTTCTTTTTCCTGGCCACCAAACAGCAGGT
TCATATTTCTCAGGAAACTTTTCAAGCATAACACCTAATAAAGGAAGAGGA
TAAGCTTTATCAAGAGCCA
(SNP base bold):
Seq ID No. 2
CTTTCTTCTGTATTTCCTTCTTTTCTTTTTCCTGGCCACCAAACAGCAGGT
TCATATTTCGCAGGAAACTTTTCAAGCATAACACCTAATAAAGGAAGAGGA
TAAGCTTTATCAAGAGCCA

QTL 2, as used herein, refers to a polymorphic genetic locus linked to genetic marker 017135_10651_5316 in pea linkage group 5 (LG5) on chromosome 3. The two alleles of marker 017135_10651_5316 at QTL 2 have the bases “A” or “G”, respectively, at position 71669603 of LG5, as set forth, respectively, in the nucleic acid sequences of Seq IDs 3 and 4. In cassette 1, QTL 2 may be homozygous for allele 1 (Seq ID 3) or be heterozygous (Seq IDs 3 and 4); while in cassette 2, QTL 2 may be homozygous for allele 2 (Seq ID 4) or be heterozygous (Seq IDs 3 and 4).

(SNP base bold):
Seq ID No. 3
TGAGATCACAGTTACTCAACATACAACTTAAATGAAATATAACGAATTAGC
ATAAAACTCAAGAGGAGGGCATACATCTTCACCAATTGAAACAGCTTCAGG
GAAGAGCCCGTGAATGAGA
(SNP base bold):
Seq ID No. 4
TGAGATCACAGTTACTCAACATACAACTTAAATGAAATATAACGAATTAGC
ATAAAACTCGAGAGGAGGGCATACATCTTCACCAATTGAAACAGCTTCAGG
GAAGAGCCCGTGAATGAGA

QTL 3, as used herein, refers to a polymorphic genetic locus linked to genetic marker 050373_32960_3169 in pea linkage group 3 (LG3) on chromosome 5. The two alleles of marker 050373_32960_3169 at QTL 3 have the bases “A” or “G”, respectively, at position 225883023 of LG3, as set forth, respectively, in the nucleic acid sequences of Seq IDs 5 and 6. In cassette 3, QTL 3 may be homozygous for allele 1 (Seq ID 5) or be heterozygous (Seq IDs 5 and 6).

(SNP base bold):
Seq ID No. 5
GTTTACATAAGATTAGAATGAATTGATCACTACTATACAGTTTTGAGAAAT
GAAATACACAAGGAATGCGTTATGTACGCAGAACAGGGAAAGGGAATCAAG
AATCGGTAGTGGAATCGAT
(SNP base bold):
Seq ID No. 6
GTTTACATAAGATTAGAATGAATTGATCACTACTATACAGTTTTGAGAAAT
GAAATACACGAGGAATGCGTTATGTACGCAGAACAGGGAAAGGGAATCAAG
AATCGGTAGTGGAATCGAT

QTL 4, as used herein, refers to a polymorphic genetic locus linked to genetic marker 029308_17474_1688 in pea linkage group 1 (LG1) on chromosome 2. The two alleles of marker 029308_17474_1688 at QTL 4 have the bases “T” or “G”, respectively, at position 106604303 of LG1, as set forth, respectively, in the nucleic acid sequences of Seq IDs 7 and 8. In cassette 3, QTL 4 may be homozygous for allele 1 (Seq ID 7) or be heterozygous (Seq IDs 7 and 8).

(SNP base bold):
Seq ID No. 7
TTTTTTGGTTCTTCTATAGACATATTCAACTAGTTTGTTTGCATCCATGGT
TCCTGTCACTGTTACTTTTCCTGTGCTAAACTCCGTCACTGCGGTTTGAAC
TCCTACAATAATCCATACA
(SNP base bold):
Seq ID No. 8
TTTTTTGGTTCTTCTATAGACATATTCAACTAGTTTGTTTGCATCCATGGT
TCCTGTCACGGTTACTTTTCCTGTGCTAAACTCCGTCACTGCGGTTTGAAC
TCCTACAATAATCCATACA

QTL 5, as used herein, refers to a polymorphic genetic locus linked to genetic marker 044073_28004_2765 in pea linkage group 5 (LG5) on chromosome 3. The two alleles of marker 044073_28004_2765 at QTL 5 have the bases “A” or “C”, respectively, at position 44420741 of LG5, as set forth, respectively, in the nucleic acid sequences of Seq IDs 9 and 10. In cassette 4, QTL 5 may be homozygous for allele 1 (Seq ID 9) or be heterozygous (Seq IDs 9 and 10).

(SNP base bold):
Seq ID No. 9
ATACCATGCAGGATTAGCTGCAGCAAGGACAGCAGTCCTTGCATTCAGTGA
TGTAGTGATACCAGCCTTGGCAATGCTAACAGTCTGTTGTTCCATAACTTC
ATGTATAGATGTACGATCA
(SNP base bold):
Seq ID No. 10
ATACCATGCAGGATTAGCTGCAGCAAGGACAGCAGTCCTTGCATTCAGTGA
TGTAGTGATCCCAGCCTTGGCAATGCTAACAGTCTGTTGTTCCATAACTTC
ATGTATAGATGTACGATCA

QTL 6, as used herein, refers to a polymorphic genetic locus linked to genetic marker 07_32684348 in pea linkage group 2 (LG2) on chromosome 6. The two alleles of marker 07_32684348 at QTL 6 have the bases “A” or “T”, respectively, at position 259389351 of LG2, as set forth, respectively, in the nucleic acid sequences of Seq IDs 11 and 12. In cassette 4, QTL 6 may be homozygous for allele 1 (Seq ID 11) or be heterozygous (Seq IDs 11 and 12).

(SNP base bold):
Seq ID No. 11
GTCCCTAATGCTGCTTATGCTGGTGGTGGCCCAAGGAGTTCATGGCCCGCA
CAGGCTCCCTCTGGCTATGGCTCTATGGGTTATGGAAACACTGCTCCTTGG
(SNP base bold):
Seq ID No. 12
GTCCCTAATGCTGCTTATGCTGGTGGTGGCCCAAGGAGTTCATGGCCCGCT
CAGGCTCCCTCTGGCTATGGCTCTATGGGTTATGGAAACACTGCTCCTTGG

QTL 7, as used herein, refers to a polymorphic genetic locus linked to genetic marker 42662_26712_871 in pea linkage group 1 (LG1) on chromosome 2. The two alleles of marker 42662_26712_871 at QTL 7 have the bases “T” or “C”, respectively, at position 410200645 of LG1, as set forth, respectively, in the nucleic acid sequences of Seq IDs 13 and 14. In cassettes 1 to 4, QTL 7 may be homozygous for allele 1 (Seq ID 13) or be heterozygous (Seq IDs 13 and 14).

(SNP base bold):
Seq ID No. 13
AGGTGGTGTTTCTGTTTTGTGTTCTTTACTTGGTCCTTTTACTTCATATGC
TGTTGGTTCTGAAGTTATTGGTATTCTTGTTAGTTTGACACTTGATTCTGA
ATCCAAAAAGAATCTTATG
(SNP base bold):
Seq ID No. 14
AGGTGGTGTTTCTGTTTTGTGTTCTTTACTTGGTCCTTTTACTTCATATGC
TGTTGGTTCCGAAGTTATTGGTATTCTTGTTAGTTTGACACTTGATTCTGA
ATCCAAAAAGAATCTTATG

QTL 8, as used herein, refers to a polymorphic genetic locus linked to genetic marker 044504_28363_461 in pea linkage group 6 (LG6) on chromosome 1. The two alleles of marker 044504_28363_461 at QTL 8 have the bases “T” or “C”, respectively, at position 167946502 of LG6, as set forth, respectively, in the nucleic acid sequences of Seq IDs 15 and 16. In cassettes 2 and 3, QTL 8 may be homozygous for allele 1 (Seq ID 15) or be heterozygous (Seq IDs 15 and 16).

(SNP base bold):
Seq ID No. 15
ACATATAATAGCACGTCGAAGATCTTCATCGTCCTTACTACAGAGCACTTG
CACATATTGTATAAGGTTTGGAAACATCTCTTTTTCCGTTGTTGATGACAA
CGGAAAAAGAGACTTTTGT
(SNP base bold):
Seq ID No. 16
ACATATAATAGCACGTCGAAGATCTTCATCGTCCTTACTACAGAGCACTTG
CACATATTGCATAAGGTTTGGAAACATCTCTTTTTCCGTTGTTGATGACAA
CGGAAAAAGAGACTTTTGT

QTL 9, as used herein, refers to a polymorphic genetic locus linked to genetic marker ER1 in pea linkage group 6 (LG6) on chromosome 1. The two alleles of marker ER1 at QTL 9 have the bases “C” or “G”, respectively, at position 175515672 of LG6, as set forth, respectively, in the nucleic acid sequences of Seq IDs 17 and 18. In cassette 1, QTL 9 may be homozygous for allele 2 (Seq ID 18) or be heterozygous (Seq IDs 17 and 18).

(SNP base bold):
Seq ID No. 17
GGTTTGCAAGGGACACAACATTTGGAAGAAGGCACTTGAGCATGTGGGCTC
AGTCACCTATTTTGTTATGGATTGTAAGGGAACTTTTGTTACATAAAATTA
ATCATACACATTAATTAAAT
(SNP base bold):
Seq ID No. 18
GGTTTGCAAGGGACACAACATTTGGAAGAAGGCACTTGAGCATGTGGGCTC
AGTGACCTATTTTGTTATGGATTGTAAGGGAACTTTTGTTACATAAAATTA
ATCATACACATTAATTAAAT

QTL 10, as used herein, refers to a polymorphic genetic locus linked to genetic marker 044835_28587_1878 in pea linkage group 1 (LG1) on chromosome 2. The two alleles of marker 044835_28587_1878 at QTL 10 have the bases “T” or “G”, respectively, at position 419557580 of LG1, as set forth, respectively, in the nucleic acid sequences of Seq IDs 19 and 20. In cassettes 1 to 4, QTL 10 may be homozygous for allele 1 (Seq ID 19) or be heterozygous (Seq IDs 19 and 20).

(SNP base bold):
Seq ID No. 19
TACATCAGTTTGAGAAAGTTACAGCAGAACTCACAACTCAAGAAGAAACTT
GCAATTTGTTATATCAACCGGAATTTCGCCAACGAGGTTTAAGTTGCTCAA
ATCCAGCAATTCAAGCAGC
(SNP base bold):
Seq ID No. 20
TACATCAGTTTGAGAAAGTTACAGCAGAACTCACAACTCAAGAAGAAACTT
GCAATTTGTGATATCAACCGGAATTTCGCCAACGAGGTTTAAGTTGCTCAA
ATCCAGCAATTCAAGCAGC

QTL 11, as used herein, refers to a polymorphic genetic locus linked to genetic marker 044855_28602_1561 in pea linkage group 1 (LG1) on chromosome 2. The two alleles of marker 044855_28602_1561 at QTL 11 have the bases “A” or “G”, respectively, at position 419560368 of LG1, as set forth, respectively, in the nucleic acid sequences of Seq IDs 21 and 22. In cassettes 1 to 4, QTL 11 may be homozygous for allele 1 (Seq ID 21) or be heterozygous (Seq IDs 21 and 22).

(SNP base bold):
Seq ID No. 21
CATTACCTCACTTGACCAAGCCTTCAACCAAGCAAAGAAGCGTAGTCAAAA
AGTTTGTGGAGTTATAATATCAAACCCTTCAAACCCTACCGGAAAATTCTT
AAATCGGGAAACACTACTT
(SNP base bold):
Seq ID No. 22
CATTACCTCACTTGACCAAGCCTTCAACCAAGCAAAGAAGCGTAGTCAAAA
AGTTTGTGGGGTTATAATATCAAACCCTTCAAACCCTACCGGAAAATTCTT
AAATCGGGAAACACTACTT

Disclosed pea plant having high protein content, or part(s) thereof are provided. The pea plant comprises a plurality of loci associated with a corresponding plurality of QTLs having a corresponding plurality of nucleic acid genetic markers that are associated with a plurality of phenotypic traits of the pea plant, wherein the phenotypic traits comprise a high protein content of the seeds of at least 25% and semi-leafless and powdery mildew resistance, and wherein the plurality of QTLs and corresponding markers comprise at least two QTLs and corresponding markers.

In certain embodiments, the QTL and marker associated with the high protein trait comprise QTL 1 with corresponding marker set forth in Seq. IDs 1 or 2; the QTL and marker associated with the semi-leafless trait comprise QTL 7 with corresponding markers set forth in Seq. IDs 13 or 14; and the QTL and marker associated with the powdery mildew resistance trait comprise QTL 9 with corresponding markers set forth in Seq. IDs 17 or 18.

In certain embodiments, the QTL and marker associated with the high protein trait comprise QTL 1 with corresponding marker set forth in Seq. IDs 1 or 2; the QTL and marker associated with the semi-leafless trait comprise QTL 7 with corresponding markers set forth in Seq. IDs 13 or 14; and the QTL and marker associated with the powdery mildew resistance trait comprise QTL 8 with corresponding markers set forth in Seq. IDs 15 or 16.

In certain embodiments, the pea plant or part thereof may further comprise a QTL and marker associated with a protein composition trait that comprise QTL 2 with corresponding marker set forth in Seq. IDs 3 or 4.

In certain embodiments, the QTL and marker associated with the high protein trait comprise QTL 3 with corresponding marker set forth in Seq. IDs 5 or 6; and QTL 4 with corresponding marker set forth in Seq. IDs 7 or 8; the QTL and marker associated with the semi-leafless trait comprise QTL 7 with corresponding markers set forth in Seq. IDs 13 or 14; and the QTL and marker associated with the powdery mildew resistance trait comprise QTL 8 with corresponding markers set forth in Seq. IDs 15 or 16.

In certain embodiments, the QTL and marker associated with the high protein trait comprise QTL 5 with corresponding marker set forth in Seq. IDs 9 or 10; and the QTL and marker associated with the semi-leafless trait comprise QTL 7 with corresponding markers set forth in Seq. IDs 13 or 14. The pea plant or part thereof may further comprise a QTL and marker associated with a protein composition trait that comprise QTL 6 with corresponding marker set forth in Seq. IDs 11 or 12.

In certain embodiments, the phenotypic traits further comprise a protein composition trait and/or a yellow cotyledon trait. In certain embodiments, the plurality of QTLs and corresponding markers comprise at least three QTLs and corresponding markers. In certain embodiments, the phenotypic traits further comprise a yellow cotyledon trait, for example, the QTLs and markers associated with the yellow cotyledon trait comprise QTL 10 with corresponding markers set forth in Seq. IDs 19 or 20; and/or QTL 11 with corresponding markers set forth in Seq. IDs 21 or 22.

In certain embodiments, the phenotypic traits comprise a high protein content of the seeds of at least 25%, or possibly a high protein content of the seeds of at least 26%. In various embodiments, the plants may be hybrids and/or the plant parts may comprise any of: a seed, an endosperm, an ovule, pollen, cell, cell culture, tissue culture, plant organ, protoplast, meristem, embryo, or a combination thereof.

FIG. 3 is a high-level schematic illustration of a breeding method 200, according to some embodiments of the invention. Breeding method 200 comprises stages of trait discovery by growing and phenotyping a broad spectrum of varieties (stage 210), trait blending by crossing the lines to mix and combine traits (stage 220), target Product Genomic Code (TPGC) discovery by associating phenotypes and genotypes using derived linkage maps (stage 230), in silico validation to suggest candidate varieties (stage 240), breeding of the candidate varieties to identify varieties with the best TPGC potential (stage 250) and genomic code (GC) discovery to identify the most stable QTLs in progeny generation(s) (stage 260), as explained in detail below.

In embodiments, pea lines were bred to reach high protein levels by collecting various pea lines worldwide, creating F2 linkage populations, applying intensive phenotyping and genotyping of thousands of pea lines, predicting of QTL's affecting the protein level trait, and establishing unique marker combinations, termed “marker cassettes” herein, to characterize novel high protein level lines found by the method and not existing in commercial or natural lines.

The breeding methodology was based on algorithms for deriving the Target Product Genomic Code (TPGC) to associate (i) the Target Product (TP) being defined in advance based on market requirements and including a set of desired attributes (traits) that are available in natural genetic variations; and (ii) the Genomic Code (GC) comprising set(s) of genomic regions that include quantitative trait loci (QTLs) that affect and are linked to the TP traits. The algorithms may be configured to calculate multiple genomic interactions and to maximize the genomic potential of specific plants for the development of new varieties. The breeding program was constructed to derive the TPGC, and then by crossing and selfing to achieve a product which contains the specific GC that corresponds to the required TPs.

Certain embodiments of the breeding process comprise stages such as: (i) Trait Discovery, in which a broad spectrum of varieties from different geographies and worldwide sources are grown and phenotyped in order to discover new traits that can potentially be combined to create a new product; (ii) Trait Blend, in which a crossing cycle is carried out based on phenotypic assumption(s), in which the different traits are mixed and combined. Initial trait cycle(s) are followed by additional cycle(s) to create F2 (and possibly higher generations) population(s) that provide the basis for algorithmic analysis for constructing the TPGC; (iii) TPGC Discovery, in which the plant(s) are phenotyped and genotyped to produce linkage map(s), discovering the relevant QTLs and deriving the TPGC; (iv) several line validation stages over several years, in which pea lines based on millions of in silico calculated variations (and/or selections) are grown and are used to defined the initial varieties; (v) Trait TPGC Blend, in which accurate crossings are performed in order to calculate the most efficient way to reach the best TPGC. The crossings are performed after in silico selection from millions of combinations, and are based, at least on part on phenotype assumptions; and (vi) Consecutive algorithm-based GC discovery stage(s) applied to F2 (or higher generation) population(s) grown in additional cycle(s).

Defining the TP for high protein level pea varieties includes the development of high throughput methods for high protein level identification. Protein level was measured using the total Kjeldahl Nitrogen method (heating the sample with concentrated sulfuric acid and optionally a catalyst to oxidize the sample and liberate the reduced nitrogen as ammonium sulfate, followed by distillation) and total amino acid analysis after acid hydrolysis. In order to screen thousands of individuals every season, NIR (near infrared) analyzers were calibrated to measure total protein, total amino acid and moisture content using a wide spectrum of pea seeds compositional analysis. For protein composition, densitometry analysis of SDS PAGE (sodium dodecyl sulfate— polyacrylamide gel electrophoresis) was used to quantify pea two major storage proteins, legumin and vicilin. In addition, in order to breed for commercial pea protein varieties, phenotypic traits such as semi-leafless, yellow cotyledon and powdery mildew resistance were also included in the target product and as part of the TPGC. In various embodiments, TPGC includes combinations of unique traits (relating to high protein levels and to other phenotypic traits) that are associated with combinations of QTLs—yielding for high protein pea.

In the following non-limiting example of the process, Trait Discovery (i) was based on germplasm including four hundred different pea lines that were obtained from the gene banks around the world. Of these, fifty different lines were used for the Trait Blend stage (ii), with crosses executed based on the potential for enrichment of genomic diversity to create new complex(es) of traits for the high protein level as the initial step for the TP-directed breeding program for high protein level pea lines. The resulted F1 hybrids were later self-crossed to create F2 linkage populations that showed phenotypic segregation. The F2 population were then planted in two different environments for discovering the TPGC (iii) that includes high protein level traits. After screening 90,000 individuals, a set of ca. 3200 representatives was selected. The selected individuals F2 was massively phenotyped for high protein level, seed color, leaf type and powdery mildew resistance components, as detailed in the following. For protein level, seed samples were tested for protein, total amino acids and moisture content using a NIR analyzer calibrated by a wide spectrum of pea seeds compositional analysis using the total Kjeldahl Nitrogen method for protein analysis and total amino acid determination in foodstuffs after acid hydrolysis using an ionic chromatography. Pea legumin and vicilin protein subunits were quantified using SDS PAGE densitometry. The measurement results were summarized into the representative high protein level trait and into the protein composition trait. Evaluation of seed color and cotyledon color was carried out by visual inspection, with the cotyledon color graded as yellow, green or mixture. Powdery mildew field resistance was evaluated by visual inspection after harvest, with plants graded as infected or not infected (resistant).

TPGC Discovery (iii) included genotyping ca. 3200 selected individual plants from 8 populations. The analysis was performed with a panel of 600 markers based on single nucleotide polymorphism (SNP) and directly designed based on the polymorphism found in the parental lines of the populations which were analyzed in depth using GenoPea™ array (Tayeh et al. 2015, Development of two major resources for pea genomics: the GenoPea™ 13.2 K SNP Array and a high-density, high-resolution consensus genetic map, The Plant Journal, Volume 84, Issue 6), Humphry et al. 2011 (Durable broad-spectrum powdery mildew resistance in pea ER1 plants is conferred by natural loss-of-function mutations in PsMLO1, Molecular Plant Pathology 12: 866-878) and independent RNA sequence analysis. The Panel was designed to maximize the chance to have the largest number of common segregate SNP's in order to create highly similar linkage maps for all observed populations. The computation of linkage maps was executed on each linkage F2 population based on the genotyping results. Linkage maps were computed with MultiPoint™, an interactive package for ordering multilocus genetic maps, and verification of maps based on resampling techniques. Discovery of QTLs that are related to high protein level was carried out with the MultiQTL™ package, based on the linkage maps that were merged by Multipoint and the F2 population phenotype data, and using multiple interval mapping (MIM). MultiQTL™ significance was computed with permutation, bootstrap tools and FDR (false discovery rate) for total analysis. The linkage maps of all eight F2 populations and the information of the high protein level traits over all genotyped plants belonging to those populations were analyzed and used to predict the QTLs in a “one trait to one marker” model, in which for all markers that constructed the linkage maps, each trait was tested independently against each one of the markers. In the provided examples, 54 markers were found to be related to protein content and to protein composition, between 2-19 markers per population. Out of these markers, four markers (linked to corresponding QTLs 1, 3, 4, 5 in Table 1) were selected to use for identifying high protein lines and two markers (linked to QTL 2 and 6 in Table 1) were found to be associated with the protein composition trait (see also Table 2). In addition, one marker (linked to QTL 7 in Table 1) was found to be associated with the semi-leafless trait, two markers (linked to QTL 10 and 11 in Table 1) were found to be associated with the yellow cotyledon trait, and two markers (linked to QTLs 8 and 9 in Table 1) were found to be associated with the powdery mildew resistance trait. In general, the populations presented different markers that related to high protein levels. However, subsets of common markers were found to be shared by multiple populations, and are referred to herein as marker cassettes. The significance and co-occurrences of the high protein level markers were evaluated using an algorithm that related the genotype-phase of each marker to respective QTLs and traits in linkage F2 in each population, for populations in different environments. The occurrence of high protein level markers in two or more linkage F2 population (repetitive markers) strengthened its significance as representative for high protein level QTL. In addition, the co-occurrence of non-repetitive and repetitive markers related to high protein level in a given population was observed for the design of the marker cassettes that provide the genetic signature for high protein level pea lines.

Following TPGC Discovery (iii), an in-silico breeding program (iv) was established to process the TPGC blend (including combinations of QTLs for different plants) to simulate and predict the genotypic states of self, cross-self and hybrid plant with respect to the QTLs and their predicted effects on each phase of the markers for the high protein level trait. The in-silico breeding program was constructed to yield millions of in silico selfing combinations, which were then bred and evaluated up to F8— to measure the potential for each of the genotyped plants to acquire the high protein level in the right combination at the right phase. The analysis resulted in identifying ca. 200 plants having the highest score for high protein level, which were thus chosen for the actual selfing and cross-selfing procedures. Under this procedure, QTLs from different population were combined to yield plants containing new and unique cassettes of QTLs and yielding high protein levels.

The high protein level pea lines were then validated as retaining the trait in the following generations by genotyping the offspring to verify they maintain the identified marker cassettes. Specifically, the parental lines of linkage F2 populations together with 190 different pea cultivars (landraces and commercial varieties) were genotyped based on high protein level markers of all populations. The cassettes detailed in Table 1 were found to wholly differentiate the developed high protein lines and the rest of the pea cultivars screened.

Disclosed pea lines that reach high protein content larger than 25%, e.g., in various lines, 26%, 27%, 28%, 30%, 35% or intermediate values (as dry weight percentage). Such high protein content allows using the disclosed pea lines for producing high protein concentrate (>65% dry weight percentage) for textured vegetable products (TVP) such as meat replacements. Moreover, advantageously, disclosed pea lines that enable the use of a sustainable and cost-effective protein enrichment process using dry fractionation and/or air classification as a processing method, which do not require large amounts of water and solvents (or even not requiring addition of any water or solvents, and having a significantly lower energy consumption) as the wet fractionation methods applied to prior art pea lines with lower protein content. Enabling dry fractionation to yield higher purity protein concentrate products also opens the possibility to use disclosed pea lines to produce highly nutritious and functional TVPs for human consumption, rather than the more common prior art use of pea concentrate for animal feed (typically 55% protein weight percent in commercial pea concentrates), due to their lower protein content and poor quality. Specifically, different disclosed pea lines were used to produce by dry fractionation texturized pea protein products having 63%, 64%, 66%, 68% and 72% protein. Generally, disclosed pea lines may be used to produce by dry fractionation texturized pea protein products having any of above 60%, above 65% or above 70% protein.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A pea plant or a part thereof that has high protein content, the pea plant comprising:
a plurality of loci associated with a corresponding plurality quantitative trait loci (QTLs) having a corresponding plurality of nucleic acid genetic markers that are associated with a plurality of phenotypic traits of the pea plant, wherein:
the phenotypic traits comprise a high protein content of the seeds of at least 25% and semi-leafless and powdery mildew resistance,
the plurality of QTLs and corresponding markers comprise at least three QTLs and corresponding markers,
the QTL and marker associated with the semi-leafless trait comprise QTL 7 with corresponding markers set forth in Seq. IDs 13 or 14, and
the pea plant or part thereof is homozygous with respect to Seq. ID 13 or heterozygous at QTL 7.

2. The pea plant or a part thereof according to claim 1, wherein:
the QTL and marker associated with the high protein trait comprise QTL 1 with corresponding marker set forth in Seq. IDs 1 or 2,
the pea plant or part thereof comprise QTL 2 with corresponding marker set forth in Seq. IDs 3 or 4,
the QTL and marker associated with the powdery mildew resistance trait comprise QTL 9 with corresponding markers set forth in Seq. IDs 17 or 18,
the pea plant or part thereof is homozygous with respect to Seq. ID 2 or heterozygous at QTL 1,
the pea plant or part thereof is homozygous with respect to Seq. ID 3 or heterozygous at QTL 2, and
the pea plant or part thereof is homozygous with respect to Seq. ID 18 or heterozygous at QTL 9.

3. The pea plant or a part thereof according to claim 1, wherein:
the QTL and marker associated with the high protein trait comprise QTL 1 with corresponding marker set forth in Seq. IDs 1 or 2,
the pea plant or part thereof comprise QTL 2 with corresponding marker set forth in Seq. IDs 3 or 4,
the QTL and marker associated with the powdery mildew resistance trait comprise QTL 8 with corresponding markers set forth in Seq. IDs 15 or 16,
the pea plant or part thereof is homozygous with respect to Seq. ID 1 or heterozygous at QTL 1,
the pea plant or part thereof is homozygous with respect to Seq. ID 4 or heterozygous at QTL 2, and
the pea plant or part thereof is homozygous with respect to Seq. ID 15 or heterozygous at QTL 8.

4. The pea plant or a part thereof according to claim 1, wherein:
the QTL and marker associated with the high protein trait comprise QTL 3 with corresponding marker set forth in Seq. IDs 5 or 6; and QTL 4 with corresponding marker set forth in Seq. IDs 7 or 8,
the QTL and marker associated with the powdery mildew resistance trait comprise QTL 8 with corresponding markers set forth in Seq. IDs 15 or 16,
the pea plant or part thereof is homozygous with respect to Seq. ID 5 or heterozygous at QTL 3,
the pea plant or part thereof is homozygous with respect to Seq. ID 7 or heterozygous at QTL 4, and
the pea plant or part thereof is homozygous with respect to Seq. ID 15 or heterozygous at QTL 8.

5. The pea plant or a part thereof according to claim 1, wherein:
the QTL and marker associated with the high protein trait comprise QTL 5 with corresponding marker set forth in Seq. IDs 9 or 10,
the pea plant or part thereof comprise QTL 6 with corresponding marker set forth in Seq. IDs 11 or 12,
the pea plant or part thereof is homozygous with respect to Seq. ID 9 or heterozygous at QTL 5, and
the pea plant or part thereof is homozygous with respect to Seq. ID 11 or heterozygous at QTL 6.

6. The pea plant or a part thereof according to claim 1, wherein the plurality of QTLs and corresponding markers comprise at least four QTLs and corresponding markers.

7. The pea plant or a part thereof according to claim 1, wherein the phenotypic traits further comprise a protein composition trait and/or a yellow cotyledon trait.

8. The pea plant or a part thereof according to claim 7, wherein the QTLs and markers associated with the protein composition trait comprise QTL 2 and/or QTL 6.

9. The pea plant or a part thereof according to claim 7, wherein the QTLs and markers associated with the yellow cotyledon trait comprise:
QTL 10 with corresponding markers set forth in Seq. IDs 19 or 20, and/or
QTL 11 with corresponding markers set forth in Seq. IDs 21 or 22, wherein:
the pea plant or part thereof is homozygous with respect to Seq. ID 19 or heterozygous at QTL 10, and/or
the pea plant or part thereof is homozygous with respect to Seq. ID 21 or heterozygous at QTL 11.

10. The pea plant or a part thereof according to claim 1, wherein the phenotypic traits comprise a high protein content of the seeds of at least 26%.

11. The pea plant or a part thereof according to claim 1, wherein the plant is a hybrid and/or the part is any of: a seed, an endosperm, an ovule, pollen, cell, cell culture, tissue culture, plant organ, protoplast, meristem, embryo, or a combination thereof.

12. A meat replacement comprising the pea plant or a part thereof according to claim 1.

13. A high protein concentrate comprising the pea plant or a part thereof according to claim 1, the high protein concentrate having >65% dry weight protein percentage.

14. A textured vegetable product (TVP) comprising the pea plant or a part thereof according to claim 1.

15. The meat replacement of claim 12, having above 60% protein, above 65% protein or above 70% protein.

16. The TVP of claim 14, having above 60% protein, above 65% protein or above 70% protein.

17. A pea plant or a part thereof that has high protein content, the pea plant comprising:
a plurality of loci associated with a corresponding plurality quantitative trait loci (QTLs) having a corresponding plurality of nucleic acid genetic markers that are associated with a plurality of phenotypic traits of the pea plant, wherein:
the phenotypic traits comprise a high protein content of the seeds of at least 25% and semi-leafless and powdery mildew resistance,
the QTL and marker associated with the high protein trait comprise QTL 1 with corresponding marker set forth in Seq. IDs 1 or 2,
the pea plant or part thereof comprise QTL 2 with corresponding marker set forth in Seq. IDs 3 or 4,
the QTL and marker associated with the semi-leafless trait comprise QTL 7 with corresponding markers set forth in Seq. IDs 13 or 14,
the QTL and marker associated with the powdery mildew resistance trait comprise QTL 9 with corresponding markers set forth in Seq. IDs 17 or 18,
the pea plant or part thereof is homozygous with respect to Seq. ID 2 or heterozygous at QTL 1,
the pea plant or part thereof is homozygous with respect to Seq. ID 3 or heterozygous at QTL 2,
the pea plant or part thereof is homozygous with respect to Seq. ID 13 or heterozygous at QTL 7, and
the pea plant or part thereof is homozygous with respect to Seq. ID 18 or heterozygous at QTL 9.

18. A pea plant or a part thereof that has high protein content, the pea plant comprising:
a plurality of loci associated with a corresponding plurality quantitative trait loci (QTLs) having a corresponding plurality of nucleic acid genetic markers that are associated with a plurality of phenotypic traits of the pea plant, wherein:
the phenotypic traits comprise a high protein content of the seeds of at least 25% and semi-leafless and powdery mildew resistance,
the QTL and marker associated with the high protein trait comprise QTL 1 with corresponding marker set forth in Seq. IDs 1 or 2,
the pea plant or part thereof comprise QTL 2 with corresponding marker set forth in Seq. IDs 3 or 4,
the QTL and marker associated with the semi-leafless trait comprise QTL 7 with corresponding markers set forth in Seq. IDs 13 or 14,
the QTL and marker associated with the powdery mildew resistance trait comprise QTL 8 with corresponding markers set forth in Seq. IDs 15 or 16,
the pea plant or part thereof is homozygous with respect to Seq. ID 1 or heterozygous at QTL 1,
the pea plant or part thereof is homozygous with respect to Seq. ID 4 or heterozygous at QTL 2,
the pea plant or part thereof is homozygous with respect to Seq. ID 13 or heterozygous at QTL 7, and
the pea plant or part thereof is homozygous with respect to Seq. ID 15 or heterozygous at QTL 8.

19. (canceled)

20. A pea plant or a part thereof that has high protein content, the pea plant comprising:
a plurality of loci associated with a corresponding plurality quantitative trait loci (QTLs) having a corresponding plurality of nucleic acid genetic markers that are associated with a plurality of phenotypic traits of the pea plant, wherein:
the phenotypic traits comprise a high protein content of the seeds of at least 25% and semi-leafless and powdery mildew resistance,
the QTL and marker associated with the high protein trait comprise QTL 5 with corresponding marker set forth in Seq. IDs 9 or 10,
the pea plant or part thereof comprise QTL 6 with corresponding marker set forth in Seq. IDs 11 or 12,
the QTL and marker associated with the semi-leafless trait comprise QTL 7 with corresponding markers set forth in Seq. IDs 13 or 14,
the pea plant or part thereof is homozygous with respect to Seq. ID 9 or heterozygous at QTL 5,
the pea plant or part thereof is homozygous with respect to Seq. ID 11 or heterozygous at QTL 6, and
the pea plant or part thereof is homozygous with respect to Seq. ID 13 or heterozygous at QTL 7.

21-28. (canceled)

29. Pea protein isolate comprising the pea plant or a part thereof according to claim 1.

30. (canceled)

31. Commodity whole or split grains comprising the pea plant or a part thereof according to claim 1.

32-48. (canceled)


SEQUENCE LISTING (22 sequences)

SEQ ID NO: 1 - DNA, 121 bp, Pisum sativum
ctttcttctgtatttccttcttttctttttcctggccaccaaacagcaggttcatatttctcaggaaacttttcaagcataacacctaataaaggaagaggataagctttatcaagagcca

SEQ ID NO: 2 - DNA, 121 bp, Pisum sativum
ctttcttctgtatttccttcttttctttttcctggccaccaaacagcaggttcatatttcgcaggaaacttttcaagcataacacctaataaaggaagaggataagctttatcaagagcca

SEQ ID NO: 3 - DNA, 121 bp, Pisum sativum
tgagatcacagttactcaacatacaacttaaatgaaatataacgaattagcataaaactcaagaggagggcatacatcttcaccaattgaaacagcttcagggaagagcccgtgaatgaga

SEQ ID NO: 4 - DNA, 121 bp, Pisum sativum
tgagatcacagttactcaacatacaacttaaatgaaatataacgaattagcataaaactcgagaggagggcatacatcttcaccaattgaaacagcttcagggaagagcccgtgaatgaga

SEQ ID NO: 5 - DNA, 121 bp, Pisum sativum
gtttacataagattagaatgaattgatcactactatacagttttgagaaatgaaatacacaaggaatgcgttatgtacgcagaacagggaaagggaatcaagaatcggtagtggaatcgat

SEQ ID NO: 6 - DNA, 121 bp, Pisum sativum
gtttacataagattagaatgaattgatcactactatacagttttgagaaatgaaatacacgaggaatgcgttatgtacgcagaacagggaaagggaatcaagaatcggtagtggaatcgat

SEQ ID NO: 7 - DNA, 121 bp, Pisum sativum
ttttttggttcttctatagacatattcaactagtttgtttgcatccatggttcctgtcactgttacttttcctgtgctaaactccgtcactgcggtttgaactcctacaataatccataca

SEQ ID NO: 8 - DNA, 121 bp, Pisum sativum
ttttttggttcttctatagacatattcaactagtttgtttgcatccatggttcctgtcacggttacttttcctgtgctaaactccgtcactgcggtttgaactcctacaataatccataca

SEQ ID NO: 9 - DNA, 121 bp, Pisum sativum
ataccatgcaggattagctgcagcaaggacagcagtccttgcattcagtgatgtagtgataccagccttggcaatgctaacagtctgttgttccataacttcatgtatagatgtacgatca

SEQ ID NO: 10 - DNA, 121 bp, Pisum sativum
ataccatgcaggattagctgcagcaaggacagcagtccttgcattcagtgatgtagtgatcccagccttggcaatgctaacagtctgttgttccataacttcatgtatagatgtacgatca

SEQ ID NO: 11 - DNA, 102 bp, Pisum sativum
gtccctaatgctgcttatgctggtggtggcccaaggagttcatggcccgcacaggctccctctggctatggctctatgggttatggaaacactgctccttgg

SEQ ID NO: 12 - DNA, 102 bp, Pisum sativum
gtccctaatgctgcttatgctggtggtggcccaaggagttcatggcccgctcaggctccctctggctatggctctatgggttatggaaacactgctccttgg

SEQ ID NO: 13 - DNA, 121 bp, Pisum sativum
aggtggtgtttctgttttgtgttctttacttggtccttttacttcatatgctgttggttctgaagttattggtattcttgttagtttgacacttgattctgaatccaaaaagaatcttatg

SEQ ID NO: 14 - DNA, 121 bp, Pisum sativum
aggtggtgtttctgttttgtgttctttacttggtccttttacttcatatgctgttggttccgaagttattggtattcttgttagtttgacacttgattctgaatccaaaaagaatcttatg

SEQ ID NO: 15 - DNA, 121 bp, Pisum sativum
acatataatagcacgtcgaagatcttcatcgtccttactacagagcacttgcacatattgtataaggtttggaaacatctctttttccgttgttgatgacaacggaaaaagagacttttgt

SEQ ID NO: 16 - DNA, 121 bp, Pisum sativum; identical to SEQ ID NO: 15

SEQ ID NO: 17 - DNA, 122 bp, Pisum sativum
ggtttgcaagggacacaacatttggaagaaggcacttgagcatgtgggctcagtcacctattttgttatggattgtaagggaacttttgttacataaaattaatcatacacattaattaaat

SEQ ID NO: 18 - DNA, 122 bp, Pisum sativum
ggtttgcaagggacacaacatttggaagaaggcacttgagcatgtgggctcagtgacctattttgttatggattgtaagggaacttttgttacataaaattaatcatacacattaattaaat

SEQ ID NO: 19 - DNA, 121 bp, Pisum sativum
tacatcagtttgagaaagttacagcagaactcacaactcaagaagaaacttgcaatttgttatatcaaccggaatttcgccaacgaggtttaagttgctcaaatccagcaattcaagcagc

SEQ ID NO: 20 - DNA, 121 bp, Pisum sativum
tacatcagtttgagaaagttacagcagaactcacaactcaagaagaaacttgcaatttgtgatatcaaccggaatttcgccaacgaggtttaagttgctcaaatccagcaattcaagcagc

SEQ ID NO: 21 - DNA, 121 bp, Pisum sativum
cattacctcacttgaccaagccttcaaccaagcaaagaagcgtagtcaaaaagtttgtggagttataatatcaaacccttcaaaccctaccggaaaattcttaaatcgggaaacactactt

SEQ ID NO: 22 - DNA, 121 bp, Pisum sativum
cattacctcacttgaccaagccttcaaccaagcaaagaagcgtagtcaaaaagtttgtggggttataatatcaaacccttcaaaccctaccggaaaattcttaaatcgggaaacactactt